(19) United States
(12) Patent Application Publication
(10) Pub. No.: US 2002/0155466 A1
(43) Pub. Date: Oct. 24, 2002

(54) Compositions and methods for the diagnosis and treatment of respiratory distress in newborn infants

(76) Inventors: F. Sessions Cole, Clayton, MO (US); Aaron Hamvas, St. Louis, MO (US); Michelle Trusgnich, St. Louis, MO (US); Lawrence M. Nogee, Baltimore, MD (US)

Correspondence Address: JOSEPH A. MAHONEY, MAYER, BROWN & PLATT, P.O. BOX 2828, CHICAGO, IL, 60690-2828, US

(21) Appl. No.: 10/007,920

(22) Filed: Dec. 7, 2001

Related U.S. Application Data
(60) Provisional application No. 60/251,917, filed on Dec. 7, 2000.

(51) Int. Cl. C12Q 1/68

(52) U.S. Cl. 435/6

(57) ABSTRACT

Single nucleotide polymorphisms associated with surfactant protein B deficiency and respiratory distress syndrome are disclosed. Compositions and methods for the diagnosis and treatment of respiratory distress syndrome in newborn infants are also disclosed.

### GOVERNMENT INTEREST

This invention was partly supported by grants from the National Institutes of Health NHLBI Grant Nos. 54187 and 65174; therefore, the government has certain rights to the invention.

### FIELD OF THE INVENTION

The present invention relates to compositions and methods for diagnosing and treating respiratory distress syndrome (“RDS”) in infants by identifying associated genetic polymorphisms in the surfactant protein B (“SPB”) gene.

### BACKGROUND OF THE INVENTION

Respiratory distress syndrome in newborn infants is the most frequent respiratory cause of death and morbidity in children under one year of age in the United States. It is also predictive of risk for chronic pulmonary diseases in childhood, including bronchopulmonary dysplasia and asthma. Survivors of respiratory distress syndrome with chronic respiratory disease consume twenty times more annualized dollars than unaffected children ($19,104 vs. $955) and 5.9% of all dollars spent on children from 0-18 years of age. Ireys et al., Expenditures For Care Of Children With Chronic Illnesses Enrolled In The Washington State Medicaid Program, Fiscal Year 1993, 100 PED. 197-204 (1997). Since the original description of surfactant deficiency by Avery and Mead in 1959 (Avery M E. Mead J., Surface Properties In Relation To Atelectasis And Hyaline Membrane Disease, 97 AMER. J. DIS. OF CHILD 517-523 (1959), respiratory distress syndrome has most commonly been attributed to developmental immaturity of the pulmonary surfactant production. The pulmonary surfactant is a mixture of phospholipids and proteins synthesized, packaged, and secreted by type II pneumocytes that line the distal airways. This mixture forms a monolayer at the air-liquid interface that lowers surface tension at end expiration of the respiratory cycle and thereby prevents atelectasis and ventilation-perfusion mismatch.

Despite improvement in neonatal survival as a result of exogenous surfactant administration, long-term respiratory morbidity and mortality have persisted in a significant fraction (5 percent to 25 percent) of affected infants. Pulmonary morbidity has been attributed to oxygen toxicity, barotrauma, developmental immaturity, and nutritional deficiencies. However, significant differences in pulmonary outcomes among developmentally similar infants with comparable exposures to oxygen, mechanical ventilation, and nutritional deficiency suggest that genetic factors contribute to pulmonary outcome. SPB deficiency was the first reported genetic cause of lethal respiratory distress syndrome in infants. Nogee L M, et al., Brief Report: Deficiency Of Pulmonary Surfactant Protein B In Congenital Alveolar Proteinosis, 328 N. ENG. J. MED. 406-410 (1993). Affected infants in the initial kindred were homozygous for a mutation that involved a one base pair deletion and three base pair at codon 121 in exon 4 of the SPB gene (121ins2). This mutation results in a frameshift and premature translation stop signal at codon 214 that accounts for the lack of protein by immunohistochemical staining and in tracheal effluent. Nogee L M, et al., A Mutation In The Surfactant Protein B Gene Responsible For Fatal Neonatal Respiratory Disease In Multiple Kindreds. 93 J. CLIN. INVEST. 1860-1863 (1994).

To determine whether respiratory distress syndrome is reliably correlated with loss of function mutations in the SPB gene, Applicants estimated the pathologic phenotype initially associated with this disease (congenital alveolar proteinosis) and the frequency of the most common mutation (121ins2) in two large, population-based cohorts. Applicants found one 121ins2 allele per 3,300 individuals from a New York cohort by molecular ascertainment and one 121ins2 allele per 1,000 individuals from a Missouri cohort by clinical ascertainment. See Cole et al., Population Based Estimates of Surfactant Protein B Deficiency, 105 PEDIATRICS (3 Pt 1) 538-41 (March 2000), which is fully incorporated herein by reference. The population frequency of the 121ins2 mutation, the consistent phenotype exhibited by infants with a homozygous genotype, and the absence of biologic redundancy for SPB function permit unambiguous counseling of parents of fetuses or infants homozygous for this mutation about disease progression, prognosis, and treatment options. However, the mutation clearly does not account for the majority of full term infants with lethal respiratory distress. Moreover, the incidence of non-lethal respiratory distress in infants caused by disorders of genetic regulation of SPB may be considerably greater than this allelic estimate suggests. Thus, intragenic polymorphisms may provide markers for genotype/phenotype correlations with clinically significant disturbances in SPB regulation.

A “SNP” refers to a “single nucleotide polymorpbism” and is the popular acronym given to a single nucleotide variance in the DNA of different people. About 90 percent of the DNA sequence variances in human DNA are SNPs. When two random human chromosomes are compared, they differ in about 1 in 1,200 nucleotides. Thus, a dipoid human may, on average, have about 3 million SNPs.

As shown in Table 1 below several investigators have attempted to find correlations with various microsatellite markers and polymorphisms linked with the SPB gene in small populations of infants with respiratory distress at birth or in limited population studies.

Table 1: Human SPB Gene Microsatellite Markers And Polymorphisms

| Polymorphisms/ | Ascertainment | |
|---|---|---|
| Markers¹ | (number of patients) | Investigators (ref) |
| C-A tandem repeats | Infants with RDS (82); infants | Floros et al., BIOCHEM JOURNAL |
| in Intron 4 | without RDS (137) | 1995; 305: 583-590 |
| | 20 unrelated individuals | Todd & Naylor, NUCL ACIDS |
| | Control white and black | RES. 1991; 19: 3756 |
| | infants (94); RDS white and | Kala et al., PED. RES. |
| | black (102) | 1998; 43: 169-177 |
| Intron 4 alleles | 103 white controls | Veletza et al., EXPER LUNG RES. |
| | 34 black controls; 69 Nigerian | 1996; 22: 489-494 |
| | controls; 40 black RDS | |
| (AAGG)ₙ marker | CEPH families (32); control | Kala et al., DIS MAR |
| alleles D2S388 | black and White infants (200); | 1997; 13: 153-167 |
| D2S2232 | black and white RDS infants | |
| GATA41E01 | (365); Nigerian adults (200) | |
| C-A bp1013 | 15 individuals in affected | Lin et al., MOL GEN METAB |
| T-C bp1580 | family | 1998; 64: 25-35 |
| Abbreviations used: | TOTAL: 671 controls; 589 | |
| ref = reference; | with RDS | |
| CEPH = Centre | | |
| d'Etude du | | |
| Polymorphisme | | |
| Humain; | | |
| RDS = respiratory | | |
| distress syndrome | | |

¹Genomic numbering

However, control populations without respiratory distress have limited the ability to truly ascertain the genetic risk of RDS associated with these microsatellite markers or polymorphisms. On the other hand, 22 clinically significant mutations in the SPB gene that unequivocally result in lethal respiratory distress have been identified. See FIG. 1 and Nogee et al., Allelic Heterogeneity in Hereditary Surfactant Protein B (SP-B) Deficiency, AM. J. RESPIR. & CRIT. CARE MED. 161(3 PT 1): 973-81 (March 2000).

The present invention is directed to newly discovered SNPs present on one or both alleles in infants having respiratory distress syndrome. The present invention can be used to assess an individual's risk towards RDS which in turn will permit development of more rational strategies for treatment of inherited lung diseases of infancy, and more accurate counseling for families whose infants are at genetic risk for development of respiratory distress at birth or during early childhood.

### SUMMARY OF THE INVENTION

Single nucleotide polymorphisms (“SNPs”) associated with SPB deficiency and RDS are disclosed herein. The present invention relates to SNPs in the SPB gene, and to a method for antenatal or postnatal identification of infants having RDS, including those homozygous for the 121ins2 mutation in the SPB gene. These polymorphisms provide the basis for convenient and reliable methods of screening for RDS, determining susceptibility of RDS, and for exploiting the therapeutic treatments of RDS.

### BRIEF DESCRIPTION OF THE DRAWINGS

FIG. 1 is a drawing illustrating the molecular structure and clinically significant mutations identified to date in the SPB gene; and

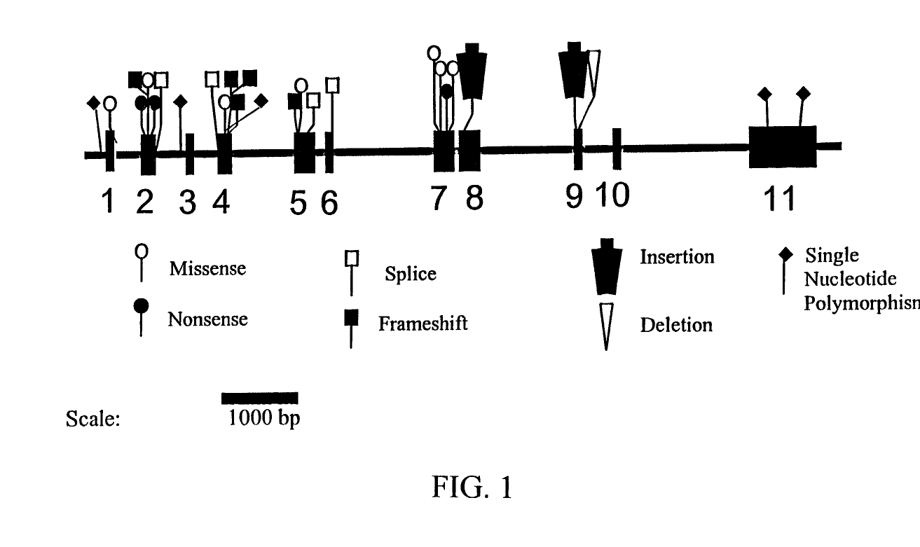

FIG. 2 is a drawing illustrating the genomic map of the single nucleotide polymorphisms of the SPB gene identified to date by the applicants.

### DETAILED DESCRIPTION

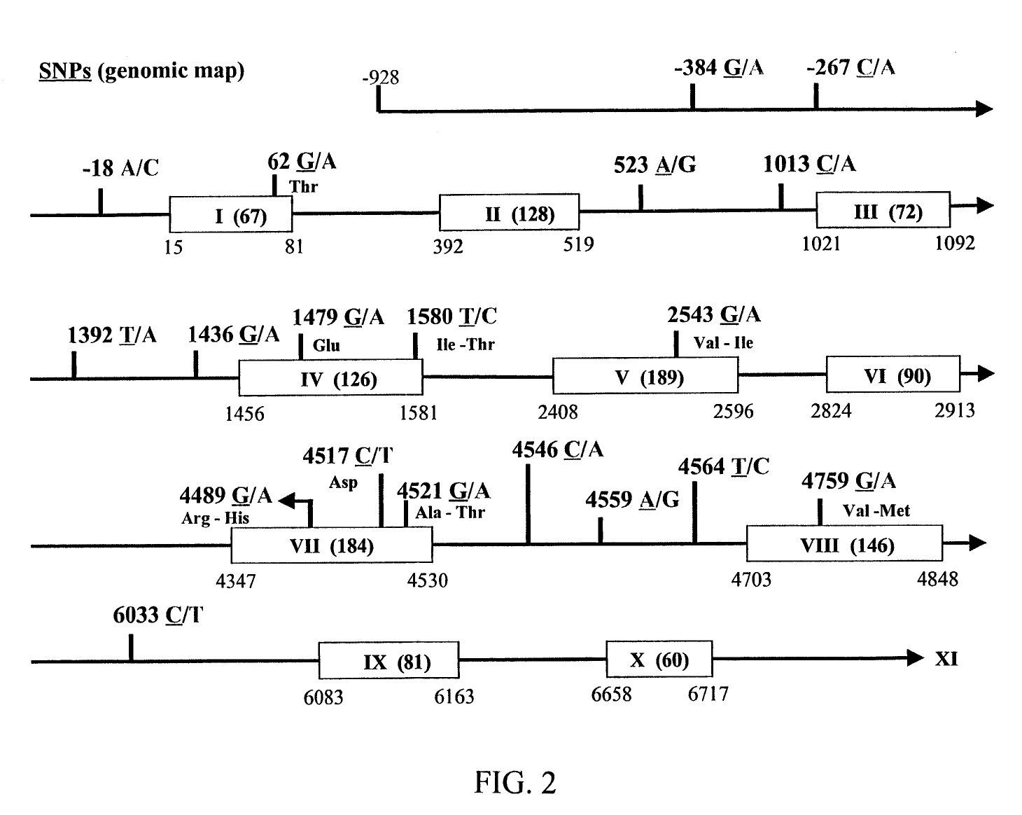

As shown in FIG. 2, applicants have identified intronic, exonic, and regulatory SNPs in the human SPB gene. These SNPs and gene fragments thereof are useful in the identification of fetal and newborn predisposition to RDS, and for the modulation of gene activity in vivo for prophylactic and therapeutic purposes. The encoded proteins of the SPB SNPs are useful, for example, as immunogens to create specific antibodies, and in drug screening for compositions wherein the presence of certain amino acid residues are indicative of RDS.

Unless defined otherwise, all technical and scientific terms used herein have the same meaning as is commonly understood by one of skill in the art to which this invention belongs. All patents and all publications mentioned herein are incorporated in their entirety by reference.

### A. Definitions

For convenience, the meaning of certain terms and phrases employed in the specification, examples, and appended claims are provided below:

“Allele,” which is used interchangeably herein with “allelic variant” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene. Alleles of a specific gene can differ from each other in a single nucleotide, or several nucleotides, and can include substitutions, deletions, and insertions of nucleotides. An allele of a gene can also be a form of a gene containing a mutation.

“Antibody” refers to polyclonal antibodies, monoclonal antibodies, entire immunoglobulin or any functional fragment. The term also encompasses fragments, like Fab and F(ab′)₂, of SPB antibodies, and conjugates of such fragments, and so-called “antigen binding proteins” (single-chain antibodies) which are based on SPB antibodies, in accordance, for example, with U.S. Pat. No. 4,704,692, incorporated herein by reference.

“Encode” in its various grammatical forms as used herein includes nucleotides and/or amino acids that correspond to other nucleotides or amino acids in the transcriptional and/or translational sense, despite the fact that they may not strictly encode for one another.

“Gene chips” (also “gene arrays” and “lab on a chip”) means the covalent attachment of oligonucleotides or cDNA directly onto a small glass or silicon chip in organized arrays. These microdevices allow rapid, microanalytical analysis of DNA or protein in a single, fully integrated system. Typically, these devices are miniature surfaces, made of silicon, glass, or plastic, which carry the necessary microdevices (pumps, valves, microfluidic controllers, and detectors) that allow sample separation and analysis.

“Homology” or “identity” or “similarity” refers to sequence similarity between two peptides or between two nucleic acid molecules. Homology can be determined by comparing a position in each sequence, which may be aligned for purposes of comparison. When a position in the compared sequence is occupied by the same base or amino acid, then the molecules are identical at that position. A degree of homology or similarity or identity between nucleic acid sequences is a function of the number of identical or matching nucleotides at positions shared by the nucleic acid sequences.

“Isolated” as used herein with respect to nucleic acids, such as DNA or RNA, refers to molecules separated from other DNAs, or RNAs, respectively, which are present in the natural source of the macromolecule. Moreover, an “isolated nucleic acid” is meant to include nucleic acid fragments, which are not naturally occurring as fragments and would not be found in the natural state. The term “isolated” is also used herein to refer to polypeptides, which are isolated from other cellular proteins and is meant to encompass both purified and recombinant polypeptides.

“Nucleic acid” refers to polynucleotides or oligonucleotides such as deoxyribonucleic acid (“DNA”), and, where appropriate, ribonucleic acid (“RNA”). The term should also be understood to include, as equivalents, analogs of either RNA or DNA made from nucleotide analogs and as applicable to the embodiment being described, single (sense or antisense) and double-stranded polynucleotides. The nucleic acids and amino acids, which occur in various amino acid sequences appearing herein, are identified according to their well-known, three letter or one letter abbreviations.

“Polymorphism” refers to the coexistence of more than one form of a gene or portion (e.g., allelic variant) thereof. A portion of a gene of which there are at least two different forms, i.e., two different nucleotide sequences, is referred to as a “polymorphic region of a gene.” A polymorphic region can be a single nucleotide, the identity of which differs in different alleles. A polymorphic region can also be several nucleotides long.

“SPB gene” means generically the surfactant protein B gene, and its alternate forms including splicing variants and polymorphisms.

“SPB nucleic acid” refers to a nucleic acid encoding a SPB protein, as well as fragments, homologs, complements, and derivatives thereof.

“SPB polypeptide” and “SPB protein” are intended to encompass polypeptides comprising the amino acid sequence, or fragments, homologs, complements, and derivatives thereof.

“SPB polymorphism or SNP” means one or more single nucleotide polymorphism for the SPB gene disclosed herein such as nucleic acids, as well as fragments, homologs, complements, and derivatives thereof.

### B. Nucleic Acid Compositions of SPB

The SPB gene has been sequenced and its regulatory regions characterized. As illustrated in FIG. 2, the gene spans approximately 10 kilobases and has 11 exons. Exons 1 through 11 encode a 2-kilobase transcript that directs the synthesis of a 381 amino acid preproprotein that is subsequently glycosylated and proteolytically processed before incorporation into pulmonary surfactant. The mature 8-kilodalton protein is encoded in exons 6 and 7. See Whitsett et al., Human Surfactant Protein B: Structure, Function, Regulation, and Genetic Disease, 75 PHYSIOL. REv. 749-57 (1995).

The SNPs of the present invention are summarized in the following Table 2, and are shown schematically in FIG. 2.

TABLE 2
SNPs in SPB Gene

| Genomic | | | Frequency | Coding | Amino | Residue |
|---|---|---|---|---|---|---|
| Location | Wild-Type | Polymorphism | (%)⁺ | Region | Acid | Location |
| −384 | G | A | 12 | Promoter | | |
| −267 | C | A | 2.5 | Promoter | | |
| −18 | A | C | 42 | Promoter | | |
| 62 | G | A | 2.5 | Exon 1 | Thr | 16 |
| 523 | A | G* | 3 | Splice | | |
| | | | | junction of | | |
| | | | | Exon 2 | | |
| 1013 | C | A | 33 | Intronic | | |
| 1392 | T | A | 1.5 | Intronic | | |
| 1436 | G | C | 1.5 | Intronic | | |
| 1479 | G | A* | <1 | Exon 4 | Glu | 97 |
| 1580 | T | C | 50 | Exon 4 | Thr -> Ile | 131 |
| 2543 | G | A* | <1 | Exon 5 | Val -> Ile | 177 |
| 4489 | G | A* | 1.5 | Exon 7 | Arg -> His | 272 |
| 4517 | C | T | 5 | Exon 7 | Asp | 281 |
| 4521 | G | A | <1 | Exon 7 | Ala -> Thr | 283 |
| 4546 | C | A | 5 | Intronic | | |
| 4559 | A | G | 16 | Intronic | | |
| 4564 | T | C | 13 | Intronic | | |
| 4759 | G | A | <1 | Exon 8 | Val -> Met | 305 |
| 6033 | C | T | 2.5 | Intronic | | |

*from preliminary data, only patients with this polymorphism had RDS
⁺from preliminary data, calculated as: # alleles with SNP/total # alleles tested

To estimate the frequency of specific SNPs in the SPB gene, applicants obtained DNA from 30 infants with RDS and 36 subjects without respiratory dysfunction. The SPB gene was amplified in 12 fragments spanning nucleotide −928 in the 5′ promoter region to nucleotide 6786 genomic bps (intron 10). Each fragment was then sequenced and the individual sequences were compared with the published sequence of the SPB gene. Deviations from the published sequence were identified as SNPs; each SNP was then analyzed with respect to the presence or absence of RDS in the subject. From a preliminary analysis these 66 subjects, four polymorphisms have been identified that only occurred in the subjects with RDS: 523 A->G, 1479 G->A, 2543 G->A, and 4489 G->A (Table 2). Thus, the presence of one or more such polymorphisms is believed to adversely change the amino acid content of SPB thereby leading to RDS. Population-based studies to determine whether these polymorphisms are over-represented in patients with RDS are underway.

Frequently, the polymorphism itself is not phenotypically expressed, but is linked to sequences that result in a disease predisposition. However, in other cases, the SNP itself may affect gene expression. The use of SNPs markers for genotyping is well documented. See, e.g., Mansfield et al., 24 GENOMICS 225-233 (1994); Ziegle et al., 14 GENOMICS 1026-1031 (1992); Dib et al., supra.

### C. Screening for RDS by Determining Presence or Absence of Polymorphic Alleles

As discussed above, several of the SNPs of the present invention may be associated with RDS. The present invention is directed to a method of screening for RDS comprising determining the presence or absence of any one of the nineteen (19) polymorphic alleles listed in Table 2, or a combination thereof, as well as others that have yet to be identified. For example, consider the SNP located at nucleotide 1580 in the genomic map shown in FIG. 2. This codon containing this nucleotide may encode an isoleucine (wild-type) or threonine (polymorphism) at amino acid residue 131 of the preproprotein. The polymorphism is referred to herein as “1580 C>T.” The nucleic acid encoding isoleucine is referred to herein as the “Ile-1580 allele.” The nucleic acid encoding a threonine is the “Thr-1580 allele.”

The following describes how this particular polymorphism may be detected in order to screen for RDS. However, one skilled in the art will appreciate that similar screening methods may be used in order to screen for any one of the polymorphisms of the present invention.

In accordance with the present invention, there are provided methods of screening for RDS comprising determining the presence or absence of polymorphic alleles of the SPB gene, wherein the presence of such an allele is indicative of RDS. Analysis may be of any convenient sample from a patient, e.g., cord blood sample, biopsy material, parental blood sample, etc. For prenatal diagnosis, fetal nucleic acid samples can be obtained from maternal blood as described in International Patent Application No. WO91/07660 to Bianchi. Alternatively, amniocytes or chorionic villi may be obtained for performing prenatal testing. Samples also include biological fluids such as tracheal lavage, blood, cerebrospinal fluid, tears, saliva, lymph, dialysis fluid, and the like; organ or tissue culture derived fluids; and fluids extracted from physiological tissues. Also included are derivatives and fractions of such fluids. The cells may be dissociated, in the case of solid tissues, or tissue sections may be analyzed. Alternatively, a lysate of the cells may be prepared.

Those skilled in the art will understand that there are numerous well known methods to detect the presence or absence of a polymorphism given the sequence information provided herein. Thus, while exemplary assay methods are described herein, the invention is not so limited. For example, in one embodiment of the invention, the presence or absence of one or more polymorphic allele in a subject's nucleic acid can be detected simply by starting with any nucleated cell sample, obtained from a subject, from which genomic DNA, for example, can be isolated in sufficient quantities for analysis. The presence or absence of the polymorphism can be determined by sequence analysis of genomic DNA, accomplished via Maxim and Gilbert (74 PROC. NATL. ACAD. Sci. USA 560 (1977)) or Sanger (Sanger et al., 74 PROC. NAT. ACAD. Sci. 5463 (1977)) or any other conventional technique.

Amplification of nucleic acid may be achieved using conventional methods, see, e.g., Maniatis, et al., MOLECULAR CLONING: A LABORATORY MANUAL 187-210 (Cold Spring Harbour Laboratory, 1982). For example, mRNA from alveolar cells can be converted to cDNA and then enzymatically amplified to produce microgram quantities of cDNA encoding SPB. Amplification, however, is preferably accomplished via the polymerase chain reaction (“PCR”) method disclosed by U.S. Pat. Nos. 4,698,195 and 4,800,159, 4,683,195 and 4,683,202 or, alternatively, in a ligase chain reaction (“LCR”) (see e.g., Landegran et al., A Ligase-Mediated Gene Detection Technique, 241 (4869) SCIENCE 1077-80 Aug. 26, 1988) and Nakazawa et al., 91 PNAS 360-364 (1994)). Alternative amplification methods include: self sustained sequence replication (Guatelli, J. C. et al., 87 PROC. NATL. ACAD. Sci. USA 1874-1878 (1990)), transcriptional amplification system (Kwoh, D. Y. et al., 86 Proc. Natl. Acad. Sci. USA 1173-1177 (1989)), Q-Beta Replicase (Lizardi, P. M. et al., 6 BIO/TECHNOLOGY 1197 (1988)), or any other nucleic acid amplification method, followed by the detection of the amplified molecules using techniques well known to those of skill in the art. These detection schemes are especially useful for the detection of nucleic acid molecules if such molecules are present in very low numbers.

The sequences complementary to the primer pairs may be separated by as many nucleotides as the PCR technique will allow. However, one of skill in the art will understand that there are practical limitations of subsequent assaying procedures, which may dictate the number of nucleotides between the sequences complementary to the primer pairs. In one embodiment, the primers are equidistant from the nucleotide(s) targeted for amplification.

The amplified nucleic acid can then be assayed by any of a variety of treatment means or methods to ascertain the genotype (and specifically the RDS genotype), including but not limited to: (1) allele-specific oligonucleotide probing, (2) differential restriction endonuclease digestion, (3) ligase-mediated gene detection (“LMGD”), (4) gel electrophoresis, (5) oligonucleotide ligation assay, (6) exonuclease-resistant nucleotides, and (7) genetic bit analysis. Additional methods of analysis would also be useful in this context, such as fluorescence resonance energy transfer (“FRET”) as disclosed by Wolf et. al., 85 Proc. Natl. Acad. Sci. USA 8790-94 (1988). Any of these or other known methods can be employed to determine the presence or absence of any one or more of the polymorphic alleles identified in Table 2 herein. Although specific examples are provided (i.e., references to the Thr-allele), the methods employed or compositions used are not intended to be limited to any one polymorphism and should be construed to encompass all polymorphisms stated herein.

### 1. Allele-Specific Oligonucleotide Probing (“ASO”)

One embodiment of the invention utilizes allele-specific oligonucleotide (“ASO”) probes for any of the polymorphic alleles, for example the Thr-1580 allele, to assay for the presence or absence of such alleles of the SPB gene. Accordingly, there is provided a method of screening for RDS, comprising assaying nucleic acid of a subject for the presence or absence of one or more polymorphic alleles of the SPB gene by contacting the nucleic acid with an allele-specific oligonucleotide probe(s) under conditions suitable to cause the probe to hybridize with nucleic acid encoding the polymorphic allele of the SPB gene, but not with nucleic acid encoding the non-polymorphic allele of the SPB gene, and detecting the presence or absence of hybridization.

Antisense oligonucleotides can be prepared as polynucleotides complementary to (a) nucleotide sequences comprising a DNA that encodes, for example, the Thr-1580 allele, or (b) nucleotide sequences comprising Thr-1580 allele messenger RNA (mRNA). For both types, the length of an antisense oligonucleotide of the present invention is not critical so long as there is no promoter sequence (for DNA) or Shine-Delgarno site (for RNA) present. Type (a) antisense oligonucleotides would be synthesized de novo, for example, based on knowledge concerning the nucleotide sequence of the genomic DNA as published. Type (b) antisense oligonucleotides could also be produced de novo (DNA or RNA), or by transforming an appropriate host organism with DNA that is transcribed constitutively into RNA which binds an SPB allele mRNA.

According to conventional ASO procedures, oligonucleotide probes are synthesized that will hybridize, under appropriate annealing conditions, exclusively to a particular amplified nucleic acid sequence that contains a nucleotide(s) that distinguishes one allele from other alleles. The probes are discernibly labeled so that when the polymorphic allele-specific oligonucleotide probe hybridizes to the sequence encoding the polymorphic allele, it can be detected, and the specific allele is thus identified.

In a preferred embodiment of the invention, the isolated nucleic acid, which is used, e.g., as a probe or a primer, is modified such as to become more stable. Exemplary nucleic acid molecules which are modified include phosphoramidate, phosphothioate, and methylphosphonate analogs of DNA (see also U.S. Pat. Nos. 5,176,996; 5,264,564; and 5,256,775).

In one embodiment of the invention, several probes capable of hybridizing specifically to allelic variants, such as single nucleotide polymorphisms, are attached to a solid phase support, e.g., a gene chip. Oligonucleotides can be bound to a solid support by a variety of processes, including lithography. For example, a chip can hold up to about 250,000 oligonucleotides. Mutation detection analysis using these chips comprising oligonucleotides is described e.g., in Cronin et al., 7 HUMAN MUTATION 244 (1996). In one embodiment, a gene chip comprises all the allelic variants of at least one polymorphic region of a gene. The solid phase support is then contacted with a test nucleic acid and hybridization to the specific probes is detected. Accordingly, the identity of numerous allelic variants of one or more genes can be identified in a simple hybridization experiment.

In another embodiment of the invention, either of the subject's amplified nucleic acid or the ASO probes can be bound onto two solid matrixes (e.g., nylon, nitrocellulose membrane, and the like) by standard techniques and then each membrane can be placed into separate hybridization reactions with an ASO probe or amplified nucleic acid, respectively. For example, if the amplified nucleic acid were bound onto a solid matrix, one hybridization reaction would utilize an oligonucleotide probe specific for the Thr-1580 allele under conditions optimal for hybridization of this probe to its complement. The other hybridization reaction would utilize an oligonucleotide specific to Ile-1580 allele under conditions optimal for hybridization of that probe to its complement. Accordingly, the ASO probes may bear the same label, but will still be distinguishable because they are hybridized in separate chambers.

This technique permits the determination of whether the subject's nucleic acid encodes the Thr-1580 allele and also whether the subject is a heterozygote or a homozygote. If an ASO probe is found to bind to subject's nucleic acid on only one membrane, then the subject is homozygous for that particular allele which the ASO probe was designed to bind. If the ASO probes are found to hybridize the subject's nucleic acid on both membranes, then the subject is heterozygous. An example of this technique applied to the detection of cystic fibrosis heterozygotes is Lemna, W. K., et al., 322 N. ENG. J. MED. 291-296 (1990).

The ASO probes of the present invention can be about 7 to about 35 nucleotides in length, preferably about 15 to 20 nucleotides in length, and are complementary to a nucleic acid sequence encoding at least the polymorphic nucleotide of SPB cDNA. Those of skill in the art will understand that other ASO probes may be designed using the sequence information provided herein. For probe design, hybridization techniques and stringency conditions, see, Ausubel, et al., (eds.) CURRENT PROTOCOLS IN MOLECULAR BIOLOGY, Wiley Intersciences, New York, sections 6.3 and 6.4 (1987, 1989).

The ASOs probes may be discernibly “labeled.” As used herein, the term “label” in its various grammatical forms refers to single atoms and molecules that are either directly or indirectly involved in the production of a detectable signal to indicate the presence of a complex (e.g., radioisotope, enzyme, chromogenic or fluorogenic substance, a chemiluminescent marker, or the like). Any label can be linked to or incorporated in an ASO probe. These atoms or molecules can be used alone or in conjunction with additional reagents. Such labels are themselves well-known in clinical diagnostic chemistry.

One of skill in the art can readily determine such conditions for hybridization based upon the nature of the probe used, factoring into consideration time, temperature, pH, and the like.

### 2. Differential Restriction Endonuclease Digestion (“DRED”)

In still another embodiment of the present invention, there is provided a method of screening for RDS, comprising assaying nucleic acid of a subject for the presence or absence of any of the polymorphic alleles of the SPB gene, for example the Thr-1580 allele, comprising cleaving a subject's nucleic acid with a restriction endonuclease, wherein the restriction endonuclease differentially cleaves nucleic acid encoding Thr-1580 allele as compared to nucleic acid encoding Ile-1580 allele, and the subject's nucleic acid comprises a sequence encoding at least nucleotide 1580 of the SPB cDNA.

DRED analysis is accomplished in the following manner. If conditions occur including (1) a particular amplified nucleic acid contains a sequence variation that distinguishes an allele of a polymorphism and (2) this sequence variation is recognized by a restriction endonuclease, then the cleavage by the enzyme of a particular nucleic acid sequence can be used to determine the allele. In accomplishing this determination, amplified nucleic acid of a subject is digested and the resulting fragments are analyzed by size or movement through a gel. The presence or absence of nucleotide fragments, corresponding to the endonuclease cleaved fragments, determines which allele is present. A restriction endonuclease suitable for use in the practice of the present invention can be readily identified by one of skill in the art.

### 3. Ligase-Mediated Gene Detection (“LMGD”)

The present invention also provides methods of screening for RDS, comprising assaying nucleic acid of a subject for the presence or absence of any polymorphic allele, e.g., the Thr-1580 allele, of the SPB gene by hybridizing the nucleic acid with a pair of oligonucleotide probes to produce a construct, wherein a first probe of the pair is labeled with a first label and a second probe of the pair is labeled with a second label, such that the first label is distinguishable from the second label, and the probes hybridize adjacent to each other. This is followed by reacting the construct with a ligase in a reaction medium, and then analyzing the reaction medium to detect the presence or absence of a ligation product comprising the first probe and the second probe.

In the course of an LMGD-type assay, a pair of oligonucleotide probes are synthesized that will hybridize adjacently to each other, for example, on a cDNA segment under appropriate annealing conditions, at the specific nucleotide that distinguishes the Thr-1580 allele from the Ile-1580 allele of SPB gene. Each ofthepairofspecific probes is labeled in a different manner, and when it hybridizes to the allele-distinguishing cDNA segment, both probes can be ligated together by the addition of a ligase. When the ligated probes are isolated from the cDNA segment, both types of labeling can be observed together, confirming the presence of the Thr-1580 allele-specific nucleotide sequence. Examples of such LMGD-type assays, which one skilled in the art may easily perform, are disclosed in Rotter et al., U.S. Pat. No. 6,008,335.

### 4. Gel Electrophoresis

In other embodiments, alterations in electrophoretic mobility will be used to identify mutations or the identity of the allelic variant of a polymorphic region in SPB genes. For example, single strand conformation polymorphism (SSCP) may be used to detect differences in electrophoretic mobility between mutant and wild type nucleic acids (Orita et al., 86 PROC. NATL. ACAD. Sci. USA 2766 (1989); see also Cotton, 285 MUTAT. RES. 125-144 (1993); and Hayashi, 9 GENET. ANAL. TECH. APPL. 73-79 (1992)). Single-stranded DNA fragments of sample and control SPB nucleic acids are denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al., 7 TRENDs GENET. 5 (1991)).

In yet another embodiment, the movement of mutant or wild-type fragments in polyacrylamide gels containing a gradient of denaturant is assayed using denaturing gradient gel electrophoresis (“DGGE”) (Myers et al., 313 NATURE 495 (1985)). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing agent gradient to identify differences in the mobility of control and sample DNA (Rosenbaum and Reissner, 265 BIOPHYS. CHEM. 12753 (1987)).

### 5. Oligonucleotide Ligation Assay (“OLA”)

In another embodiment, identification of the allelic variant is carried out using an oligonucleotide ligation assay (“OLA”), as described, e.g., in U.S. Pat. No. 4,998,617 and in Landegren et al., 241 SCIENCE 1077-1080 (1988). The OLA protocol uses two oligonucleotides, which are designed to be capable of hybridizing to abutting sequences of a single strand of a target. One of the oligonucleotides is linked to a separation marker, e.g., biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al., have described a nucleic acid detection assay that combines attributes of PCR and OLA (Nickerson, D. A. et al., 87 PROC. NATL. ACAD. Sci. U.S.A. 8923-8927 (1990)). In this method, PCR is used to achieve the exponential amplification of target DNA, which is then detected using OLA. Several techniques based on this OLA method have been developed and can be used to detect specific allelic variants of a polymorphic region of a SPB gene. For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in Tobe et al., 24 Nucleic Acids Res. 3728 (1996), OLA combined with PCR permits typing of two alleles in a single microtiter well. By marking each of the allele-specific primers with a unique hapten, i.e., digoxigenin and fluorescein, each OLA reaction can be detected by using hapten specific antibodies that are labeled with different enzyme reporters, alkaline phosphatase, or horseradish peroxidase. This system permits the detection of the two alleles using a high throughput format that leads to the production of two different colors.

### 6. Exonuclease-Resistant Nucleotides

In one embodiment, the single base polymorphism can be detected by using a specialized exonuclease-resistant nucleotide, as disclosed, e.g., in Mundy, et al. U.S. Pat. No. 4,656,127. According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment of the invention, a solution-based method is used for determining the identity of the nucleotide of a polymorphic site. Cohen, D. et al. (French Patent 2,650,840; PCT Appln. No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

### 7. Genetic Bit Analysis (GBA™)

An alternative method, known as GBA™ is described by Goelet, P. et al. (PCT Appln. No. 92/15712). The method of Goelet, P. et al., uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al., (French Patent No. 2,650,840; PCT Appln. No. WO91/02087) the method of Goelet, P. et al., is preferably a heterogeneous phase assay, in which the primer or the target molecule is immobilized to a solid phase.

### 8. Protein Truncation Test

For polymorphisms that produce premature termination of protein translation, the protein truncation test offers an efficient diagnostic approach (Roest, et. al., 2 HUM. MOL. GENET. 1719-21 (1993); van der Luijt, et. al., 20 Genomics 1-4 (1994)). For this test, RNA is initially isolated from available tissue and reverse-transcribed, and the segment of interest is amplified by PCR. The products of reverse transcription PCR are then used as a template for nested PCR amplification with a primer that contains an RNA polymerase promoter and a sequence for initiating eukaryotic translation. After amplification of the region of interest, the unique motifs incorporated into the primer permit sequential in vitro transcription and translation of the PCR products. Upon sodium dodecyl sulfate-polyacrylamide gel electrophoresis of translation products, the appearance of truncated polypeptides signals the presence of a mutation that causes premature termination of translation. In a variation of this technique, DNA (as opposed to RNA) is used as a PCR template when the target region of interest is derived from a single exon.

### 9. Antibody Binding

Screening may also be based on the functional or antigenic characteristics of the protein as is well known in the art. Immunoassays designed to detect predisposing polymorphisms in SPB proteins may be used in screening. Antibodies specific for SPB polymorphisms may be used in screening immunoassays. A reduction or increase in neutral SPB and/or presence of RDS associated polymorphisms is indicative that RDS is SPB-associated.

Thus, in one exemplification, the invention further includes antibodies, which are capable of binding SPB encoded by Thr-1580 allele, but not to SPB encoded by Ile-I 580 allele. Such an antibody may be easily produced by one of skill in the art by preparing a peptide, protein conjugate that is specific to the unique amino acid in SPB encoded by Thr-1580 allele and immunizing an animal. The invention includes the hybridoma cell line, which produces the antibody of the same specificity, the antibody produced by the hybridoma cell line and the method of production.

Antibodies raised against the Thr-1580 allele or SPB encoded by the Thr-1580 allele are expected to have utility in the diagnosis, prevention, and treatment of RDS. For therapeutic applications, the antibodies employed can be humanized, monoclonal antibodies. Antibodies against other polymorphisms may also be raised.

The above-described antibodies can be prepared employing standard techniques, as are well known to those of skill in the art, using any polymorphic allele or SPB encoded by the polymorphic allele, or fragments thereof, as antigens for antibody production.

To enhance the specificity of the antibody, the antibodies may be purified by immunoaffinity chromatography using solid phase-affixed immunizing polypeptide. The antibody is contacted with the solid phase-affixed immunizing polypeptide for a period of time sufficient for the polypeptide to immunoreact with the antibody molecules to form a solid phase-affixed immunocomplex. The bound antibodies are separated from the complex by standard techniques.

The antibody so produced can be used, inter alia, in diagnostic methods and assay methods to detect the presence or absence of nucleic acid encoding one or more polymorphic allele. See generally Rose et al., Manual of Clinical Laboratory Immunology (1997).

The SPB antibodies can also be used for the immunoaffinity or affinity chromatography purification of SPB biological materials. In addition, an SPB antibody according to the present invention can be used in mammalian therapeutic methods, preferably human, to neutralize or modulate the effect of a polymorphic allele.

Antibodies against SPB encoded by the Thr-1580 allele can also be employed in the generation, via conventional methodology, of anti-idiotypic antibodies (antibodies that bind an anti-SPB allele antibody), e.g., by the use of hybridomas as described above. See, for example, U.S. Pat. No. 4,699,880. Such anti-idiotypic antibodies could be used to sequester anti-Thr-1580 SPB antibodies in an individual, thereby treating or preventing pathological conditions which may be associated with an immune response whereby Thr-1580 allele is recognized as “foreign” by the immune system of the individual.

### D. Susceptibility to RDS

Those skilled in the art will appreciate that any of the foregoing inventive methods may be used not only to screen for RDS, but also to predict a subject's susceptibility to RDS. There is general agreement that genetics are important in a person's susceptibility to RDS. Evidence supporting this conclusion include consistent ethnic differences which cross different geographic areas, dramatic familial aggregation, existence of genetic syndromes that feature RDS, higher monozygotic than dizygotic twin concordance rates, lack of increased frequency in spouses, affected relatives separated in space and time, and associations between RDS and genetic markers. Thus, any of the inventive methods for screening for RDS may also be used as an initial screening tool to predict a subject's susceptibility to RDS.

These methods for determining susceptibility to RDS are particularly useful in combination with a subject's family history of RDS. For example, a parent who experienced RDS as a newborn and who has a family history of RDS, may well have a child who is susceptible to RDS. To alleviate the concern of the parent, and to take any preventative measures which might prevent onset, one of the many inventive methods provided herein can be used to determine whether the child is a carrier of one or more the polymorphic alleles identified herein.

Similarly, the screening methods provided herein are preferably used in combination with existing methods for diagnosing RDS (e.g., radiological and biochemical) to maximize a confidence in the ultimate diagnosis regarding RDS.

### E. Kits

Kits for use in screening for RDS and screening for susceptibility to RDS are also provided by the present invention. Such kits can include all or some of the reagents, primers, probes, antibodies, and antisense oligonucleotides described herein for determining the presence or absence of nucleic acid encoding one or more polymorphic allele, or for treatment of RDS. Kits of the present invention may contain, for example, restriction endonuclease; one or more labeled oligonucleotide probes that distinguish nucleic acid encoding the relevant nucleotides of SPB cDNA; ligase; polymorphic allele-specific oligonucleotide probe; primer for amplification of nucleic acid encoding the relevant nucleotide of SPB cDNA; means for amplifying a subject's nucleic acid encoding the cDNA; neutrophil, alkaline phosphatase coupled goat anti-human gamma chain specific antibody; fluorescein-labeled goat anti-human gamma chain specific antibody; anti-human gamma chain specific antibody; antisense oligonucleotides; antibody specific for, or which binds the polymorphic allele; or combinations of any of the above.

These suggested kit components may be packaged in a manner customary for use by those of skill in the art. For example, these suggested kit components may be immobilized on a solid matrix or provided in solution or as a liquid dispersion or the like.

### F. Methods of Treatment

The present invention provides for both prophylactic and therapeutic methods of treating a subject having or at risk of having RDS. Subjects at risk for such a disease can be identified by a diagnostic or prognostic assay as described herein. Administration of a prophylactic agent can occur prior to the manifestation of symptoms characteristic of the SPB disruption, such that development of RDS is prevented or, alternatively, ameliorated in its progression. In general, the prophylactic or therapeutic methods comprise administering to the subject an effective amount of a compound, which is capable of augmenting a wild-type SPB activity or antagonizing a mutant (defective) SPB activity.

It is to be understood that although the invention herein described is only illustrative. None of the embodiments shown herein are limiting. It is apparent to those skilled in the art that modifications and adaptations can be made without departing from the scope of the invention as defined by the claims appended.

We claim:

1. A method for screening respiratory distress syndrome in a mammalian subject, comprising:
a) obtaining a sample from the subject;
b) preparing the sample for analysis by isolating at least one of DNA, RNA, or protein from the sample; and
c) determining the presence or absence of at least one SPB polymorphism associated with the syndrome within the sample by analyzing the isolated DNA, RNA, or protein using probes specific for the polymorphism.

2. A method of predicting a mammalian subject's susceptibility to respiratory distress syndrome, comprising:
a) providing
i) a sample from the subject, wherein the sample comprises nucleic acid, the nucleic acid comprising a SPB gene, and
ii) a treatment means of at least one of: allele-specific oligonucleotide probing, differential restriction endonuclease digestion, ligase-mediated gene detection, gel electrophoresis, oligonucleotide ligation assay, exonuclease-resistant nucleotides, genetic bit analysis and fluorescence resonance energy transfer;
b) treating the sample with the treatment means under conditions such that a genotype for respiratory distress syndrome is detected if present, wherein the genotype comprises a genotype homozygous for at least one allele of a plurality of polymorphic sites of the SPB gene listed in Table 2; and
c) detecting the respiratory distress syndrome genotype if present, wherein the presence of the genotype is indicative of the subject's susceptibility to respiratory distress syndrome.

3. The method of claim 2, wherein said sample is blood.

4. An isolated and purified nucleic acid of at least one of a plurality of single nucleotide polymorphisms listed in Table 2 and FIG. 2.
